(19) United States
(12) Patent Application Publication
(10) Pub. No.: US 2007/0270686 A1
(43) Pub. Date: Nov. 22, 2007

(54) Apparatus and methods for using inertial sensing to navigate a medical device

(76) Inventors: Rogers C. Ritter, Charlottesville, VA (US); Raju R. Viswanathan, St. Louis, MO (US)

Correspondence Address: Kevin Pumm, Suite 400, 7700 Bonhomme, St. Louis, MO, 63105, US

(21) Appl. No.: 11/800,064

(22) Filed: May 3, 2007

Related U.S. Application Data
(60) Provisional application No. 60/797,252, filed on May 3, 2006.

(51) Int. Cl. A61B 5/05

(52) U.S. Cl. 600/424

(57) ABSTRACT

A system for remotely navigating a medical device in an operating region in a subject. An inertial sensing device in the medical device distal end has one or more inertial sensors that provide information for locating the medical device. A controller is operable to control movement of the medical device based on the locating information to navigate the device distal end to a target point.

### CROSS-REFERENCE TO RELATED APPLICATIONS

This application claims benefit of U.S. Provisional Patent Application Ser. No. 60/797,252, filed May 3, 2006, the entire disclosure of which is incorporated by reference.

### FIELD

The present invention relates to navigating medical devices such as catheters in the body of a subject and more particularly to using inertial sensing to help control navigation of a medical device to target points within the subject.

### BACKGROUND

Several systems are available which allow a physician or other medical professional to navigate a medical device such as a catheter, guide wire, sheath, or endoscope inside a subject's body. The distal end of a device can be steered, for example, by mechanically manipulating controls on the device proximal end. Magnetic navigation systems also have been developed which allow a physician to use the field of an external source magnet to orient the distal end of a medical device inside a subject. Other means by which a physician can orient the distal end of a medical device include electrostrictive elements incorporated into the medical device and hydraulic actuation.

Various computational and imaging methods may be used to determine the position of a medical device being navigated within an operating region in a subject's body. Fluoroscopic and other imaging techniques are commonly used to aid the physician in visualizing the operating region. Two limitations of fluoroscopy are respectively the projection nature of the imaging modality and the high patient and/or attendant x-ray radiation doses. It is desirable, of course, to determine the current position and orientation (“localization”) of a medical device distal end with speed and precision during a medical procedure. Accurate and frequently provided localization information provides useful feedback during device navigation, reduces navigation times, and increases intervention success rates.

### SUMMARY

The present invention, in one aspect, is directed to a method of navigating a medical device in an operating region of a subject. Accelerations and orientations of the device are sensed in a substantially continuous manner over time. The instantaneous sensed orientations and accelerations are used to determine by process of integration and sampling a time series of current orientation and position for the device. The current localization information is used to navigate the device to a target point within the subject.

In one aspect of the invention, various methods for controlling or operating a remote navigation system that controls the position of a medical device in an operating region are provided. One method for controlling a medical device within a subject comprises operating the remote navigation system to change the position of the medical device, and processing signals from at least one inertial sensor associated with the medical device to determine the change in position from the initial position. The method further includes comparing the determined change in position with the desired position, and repeating the steps until the current position is within a predetermined value of the desired position.

In another aspect, the invention is directed to a system for remotely navigating a medical device in an operating region in a subject. An inertial sensing system includes at least one sensing component comprising one or more inertial sensors that provide information for locating the medical device that incorporates the sensing component(s). Generally, means provided for inertial guidance comprise gyroscope(s) for the determination of three reference angles and three accelerometers. The gyroscope(s) establish an instantaneous reference frame for the orientation of the three accelerometers. The accelerometers measure velocity changes in each of these instantaneous reference frame directions. The sensed accelerations and orientations are used to determine through a first integration an instantaneous velocity, and through a second integration, an instantaneous position for the device with respect to a subject fixed reference frame, and are used to navigate the device. A controller is operable to control movement of the medical device based on the time series of localization data.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1-A is a schematic diagram of a system for navigating a medical device in an operating region of a subject in accordance with one implementation of the invention;

FIG. 1-B shows in more details an inertial sensing component embedded near the distal end of a medical device;

FIG. 2 presents schematic diagrams showing three angles (FIGS. 2-A, 2-B, 2-C) that define the instantaneous orientation of a medical device tip with respect to a fixed reference frame and also shows (FIG. 2-D) three accelerometers to measure instantaneous accelerations in the device tip frame;

FIG. 3 is a schematic diagram showing the use of more than one set of inertial sensing components at the distal end of a medical device;

FIG. 4 is a flow diagram of a controlled method of navigating a medical device in an operating region in accordance with one implementation of the invention;

FIG. 5-A is a block diagram of a magnetic navigation system in accordance with one implementation of the invention; FIG. 5-B shows in more details an inertial sensing component embedded near the distal end of a magnetic navigation medical device;

FIG. 6-A is a block diagram of a magnetic resonance imaging and magnetic navigation system in accordance with one implementation of the invention;

FIG. 6-B shows in more details an inertial sensing component embedded near the distal end of a medical device designed for magnetic navigation;

FIG. 7-A is a block diagram of a mechanical navigation system in accordance with one implementation of the invention;

FIG. 7-B shows in more details an inertial sensing component embedded near the distal end of a mechanical navigation medical device;

FIG. 8-A is a block diagram of an electrostrictive navigation system in accordance with one implementation of the invention;

FIG. 8-B shows in more details an inertial sensing component embedded near the distal end of an electrostrictive navigation medical device;

FIG. 9-A is a diagram of an hydraulic navigation system in accordance with one implementation of the invention;

FIG. 9-B shows in more details an inertial sensing component embedded near the distal end of an hydraulic navigation medical device.

### DETAILED DESCRIPTION OF EMBODIMENTS

In this invention, micro electromechanical systems (MEMS) and devices allow implementation of inertial navigation systems within a medical device, or within the tip of a medical device, such as a catheter, sheath, endoscope, or other minimally invasive interventional tools.

In various implementations of the present invention, one or more inertial sensors may be used in navigating a catheter, endoscope, or other medical device in an operating region of a subject during a medical procedure. Inertial sensing may be used, for example, in connection with magnetic, electrostrictive, hydraulic and/or mechanical navigation of medical devices. MEMS devices according to technologies known in the art allow implementation of relatively complex electromechanical systems on a spatial scale as small as a few tenths of a micro-meter. Such MEMS devices are particularly suitable for use as imbedded systems on small medical interventional tools subject to a number of environment and safety constraints, such as catheters, guide wires, sheaths, and endoscopes.

Inertial sensors may be used in some embodiments to navigate a medical device in a closed-loop manner as further described below. It will be appreciated that such loops could be configured to incorporate various servo-control methods, for example, applying gains optimized to improve signal-to-noise ratios given known signal and noise dynamic ranges, implementing statistical methods to reduce drift, or using various imaging or remote sensing means of feedback control. The accuracy of inertial navigation equipment cannot be improved indefinitely due to basic mechanical limitations. Inertial sensing device errors are cumulative over time; however it is known in the art that these limitations and associated errors can be reduced by several orders of magnitude by computer-directed statistical filtering. As an example, Kalman filtering techniques are known in the art to allow weighting of the incoming data as a function of their expected quality. Regular re-calibrations, or fixes, of a “dead-reckoning” navigation system, allow both zeroing out residual errors and improving statistical prediction models.

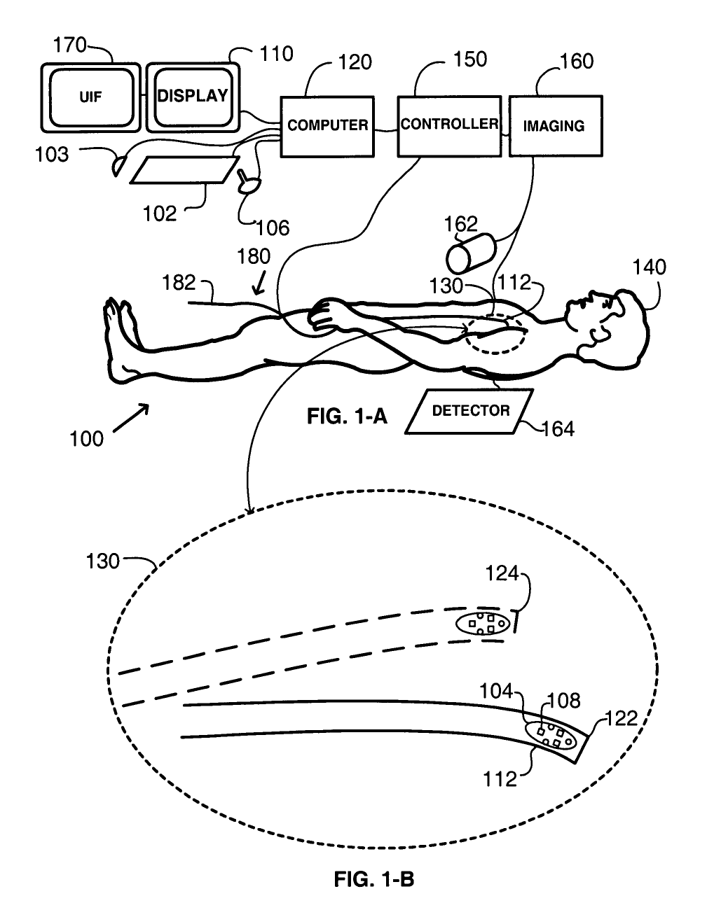

One embodiment of a system for navigating a medical device in an operating region of a subject is indicated generally in FIG. 1 by reference number 100. The system 100 includes an elongated medical device 180 comprising a proximal end 182 and a distal end 112, said distal end being navigated in an operating region 130 of a subject 140. The distal end 112 comprises an inertial sensing component 104 having one or more inertial sensors 108 such as gyroscopes and accelerometers that provide information for locating a medical device 112. A controller 150 is operable to control movement of the medical device 112 based on the localization information. In the present embodiment, the sensing device 104 includes six inertial sensors 108 configured to sense the instantaneous orientation of a local reference frame comprising three axes with respect to fixed subject axes, and configured to sense acceleration of a distal tip 122 of the medical device 112 with respect to the three local reference frame instantaneous directional axes. It should be noted, however, that embodiments also are contemplated in which fewer or more than six sensors per component, and/or more than one sensing component, may be provided. Where appropriate benchmark inputs are available for position and orientation of the medical device 112, the sensed acceleration may be integrated over an appropriate time period to obtain velocity and direction of movement of the medical device 112. In turn, the calculated velocities may be integrated to obtain a position of the device 112. A typical time period over which the foregoing integrations may be performed corresponds to part or all of the navigation intervention, or that part of the intervention following localization calibration (or “fix” in celestial navigation).

The sensed accelerations thus may be used to determine a current position of the medical device 112 in a subject operating region 130. For example, where the controller 150 has received information describing an initial position 124 and/or orientation of the device distal tip 122, the controller may process signals from at least one of the sensors 108 to determine a current position of the distal tip 122 relative to the initial position 124. The current position can be used by the controller 150 to navigate the medical device 112 in the operating region. For example, the controller 150 may compare the current distal tip position to a desired position and move the tip 122 toward the desired position and/or orientation. Computer 120 takes inputs from the user through a keyboard 102, mouse 103, joystick 106, or other input devices, such as a graphical user interface (UIF) 170, and displays information regarding the navigation on display 110. Further, the system comprises an imaging component 160, for example an x-ray fluoroscopy image chain comprising an x-ray tube 162 and an x-ray detector 164.

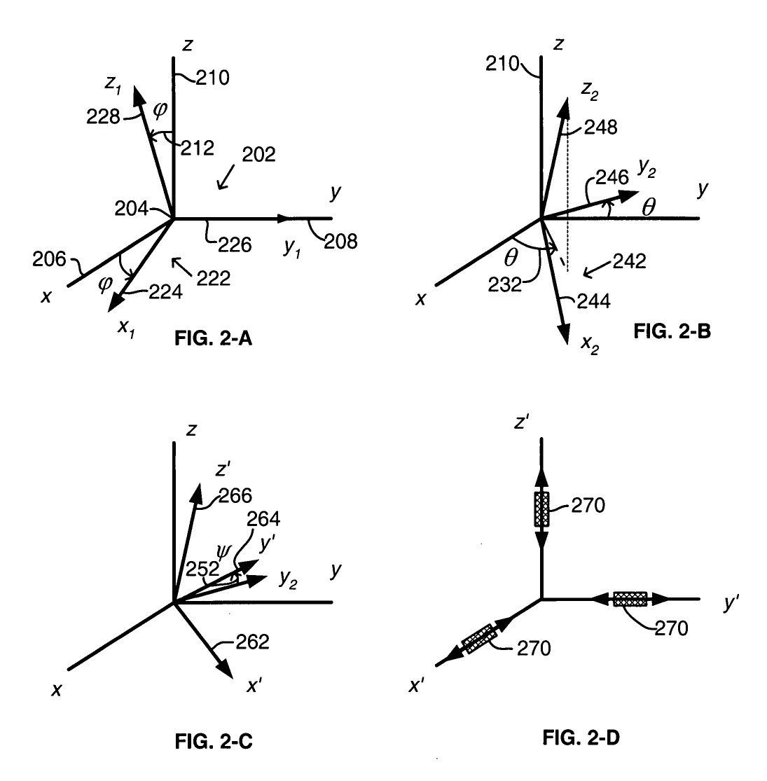

The sensed accelerations along axes of known orientations at a given time allow determination of the local, incremental, device advance. Axes orientations are given instantaneously by the gyroscope sensors of the inertial navigation MEMS component(s). Time-integration of these data time series provide localization information in the subject reference frame, and allow controlled navigation of the medical device to specific target points. A number of coordinate transformations can be used to express the coordinates of two co-centered orthogonal coordinate systems. FIG. 2 illustrates one such transformation using angles φ, θ, and ψ. Given subject fixed coordinate system 202 (O 204, x 206, y 208, z 210), FIG. 2-A, angle φ 212 describes a rotation with respect to axis y 208, leading to intermediate referential 222 (x₁ 224, y₁=y 226, z₁ 228). Angle θ 232 describes a rotation with respect to axis z 210 to intermediate referential 242 (x₂ 244, y₂ 246, Z2 248), FIG. 2-B. Finally, rotation of angle ψ 252 with respect to axis Z₂ 248 leads from (x₂, y₂, z₂) to rotated reference system 260 (x′ 262, y′ 264, z′=z₂ 266), FIG. 2-C. FIG. 2-D schematically illustrates the use of three accelerometers 270 with respect to each local device axes (x′, y′, z′). Three gyroscopes (not shown) allow tracking the instantaneous orientation of the inertial component reference frame (x′, y′, z′) with respect to the fixed subject reference frame (x, y, z). The original mechanical gyroscope in principle consists of a rapidly spinning wheel set in a framework that permits it to tilt freely in any direction; the wheel momentum causes it to retain its attitude when the framework is tilted, therefore allowing determination of relative orientation over time. More recent solid-state implementations based on MEMS technologies have used generation of standing waves and the detection and analysis of changes in the waveform to provide change of direction information, or other technologies suitable for miniaturization.

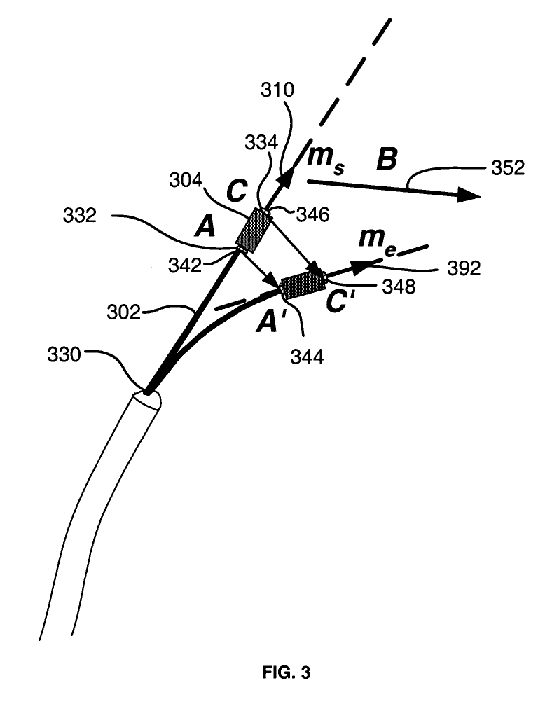

Inertial systems reliability is increased by use of more than one set of inertial sensing components. Additionally, an implementation using multiple sets provide additional data, possibly presenting redundancies, that can be combined and analyzed to reduce the effects of time-dependent errors, such errors being stochastic in nature and typically independent from one sensing component to the next. FIG. 3 illustrates the use of two sets of inertial sensing components A and C placed at either end of a magnetic guide wire 302 distal tip magnet 304. The components are located respectively proximally 332 and distally 334 the magnetic tip 304. FIG. 3 shows motion of the magnetic tip moment through a time interval Δt from an initial localization 310 to a subsequent localization 320. The respective motions of the sensors from A 342 to A′ 344 and from C 346 to C′ 348 can be tracked over time in the interval Δt. As the magnet tip 304 can be considered rigid, the respective positions and orientations at the two sensor components are correlated; analysis over time of the sensors data allows noise and drift reduction by use of digital signal processing methods such as least-squares estimation, statistic modeling, and similar techniques. Generally, this approach can be extended to a case where the medical device properties along the segment joining two sensor sets are known or can be modeled. Further, additional MEMS strain sensors on opposite sides of the medical device could allow measurement of differential strains or forces, and thus through device modeling lead to an estimate of the local device curvature as a function of applied torques and forces.

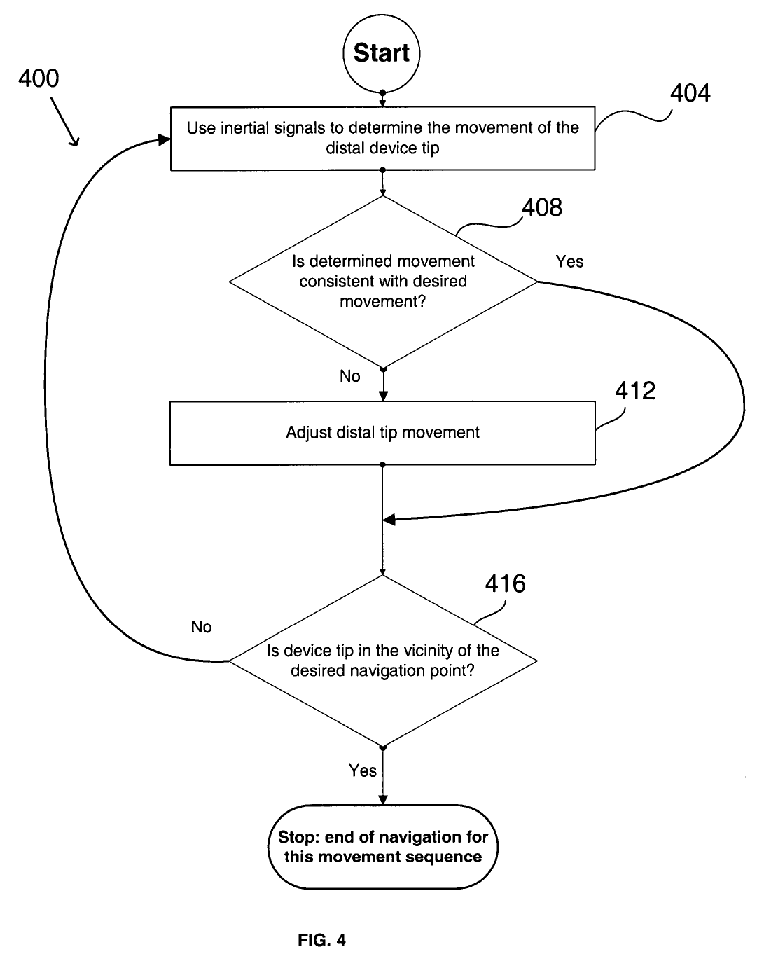

Referring again to FIG. 1, it can be appreciated that acceleration, velocity and positional information provided by the inertial sensors 108 and associated system may be used in various ways to locate and control the medical device 112. For example, one closed-loop method of navigating a medical device in an operating region that may be performed by the controller 150 is indicated generally in FIG. 4 by reference number 400. In step 404, sensor 108 signal(s) are used to determine how fast and in what direction the distal tip 122 may be moving. In step 408, the controller 150 compares the determined movement with a desired movement, e.g., movement of the tip 122 consistent with a planned path previously input to the system 100 by a physician. If the determined movement is not consistent with the desired movement, then in step 412 the controller 150 adjusts movement of the distal tip 122. For example, the controller may cause the tip to move faster or slower and/or to move closer to the planned path. In step 416 it is determined whether position and orientation of the tip 122 are within a predetermined vicinity of their desired values. If yes, the method terminates. If no, control is returned to step 404. In specific implementations, real-time physician input may be incorporated into the loop of FIG. 4.

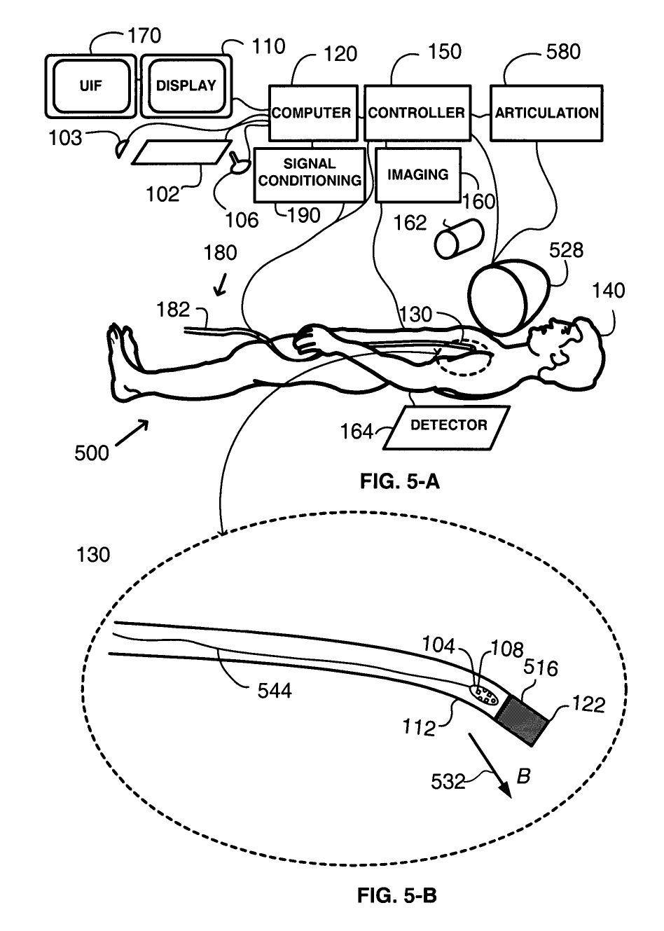

One embodiment of a system for magnetically navigating a medical device, e.g., a catheter, is indicated generally in FIG. 5 by reference number 500. An inertial sensing component 104 is provided at or near the medical device tip 122. The sensing component comprises inertial sensors 108 for sensing six parameters, for example including three gyroscopes and three accelerometers, each configured to provide an orientation or acceleration signal for one of three angles or one of three directional axes. The system 500 is a magnetic navigation system and the catheter tip 122 includes one or more magnets 516. A physician uses a graphical user interface (UIF) 170 and computer 120 to control one or a multiplicity of magnetic field source(s) 528 and to navigate the device tip 122 in a magnetic field 532 produced by the source(s) 528. The interface 170 may include a keyboard 102, mouse 103, joystick 106, and/or other device to input instructions to the computer 120. The interface 170 also may include a display 110 whereby the physician may monitor navigation of the device 112. An imaging apparatus 160 processes signals from the computer 120 and may display images of a subject operating region 130 in which the catheter 112 is being navigated. Signal leads 544 extend along the device 112 and preferably are embedded in the device wall. The leads 544 carry the six orientation and acceleration signals from the sensors 108 to the computer 120 via signal processing or conditioning components 190. The conditioning components 190 may include a computer with a preconditioning circuit to reduce data noise or to provide impedance matching. An integration of each of the three acceleration signals over an appropriate time period and with respect to time-varying device axes yields velocity magnitude and direction for the device 112. Additionally, for each sensor 108 an integration of the velocity with respect to time-varying axes yields a position of that sensor component and hence of the device 112.

Each integration involves three arbitrary constants, one for each of the three dimensions, for a total of six such constants. The constants can be established at the subject bed, preferably before insertion of the device 112, and when the catheter tip 122 is at rest. Using the inertial sensing device 104 to provide a localization sequence in a navigation procedure typically leads to a summation of small errors. Accordingly, recalibration of the six constants of integration may be performed occasionally after comparing a location determined by the sensing device 104 with one or more fiducial landmarks. Comparisons to such landmarks may be accomplished, for example, using fluoroscopic imaging. However, in some applications, it may be desirable to use the sensing device 104 to locate the catheter 112 for navigation without using x-rays. In the embodiments described below, comparisons of locations determined by the sensing device 104 to landmarks could be accomplished for some types of medical procedures by using ultrasound. For example, in cardiac procedures, ultrasound sensors inserted in the bronchial cavity could be used for imaging and localization of the device and of the inertial sensor(s) 108 at the catheter tip 122 relative to landmark features, either of the body, or artificial reference ones located on the chest.

Information from the sensing component 104 can be used to provide system feedback in various ways. For example, in the implementation shown in FIG. 5, control feedback could be used by a navigation program of the magnetic navigation system 500 to cause the catheter 112 to follow a planned path input by a physician.

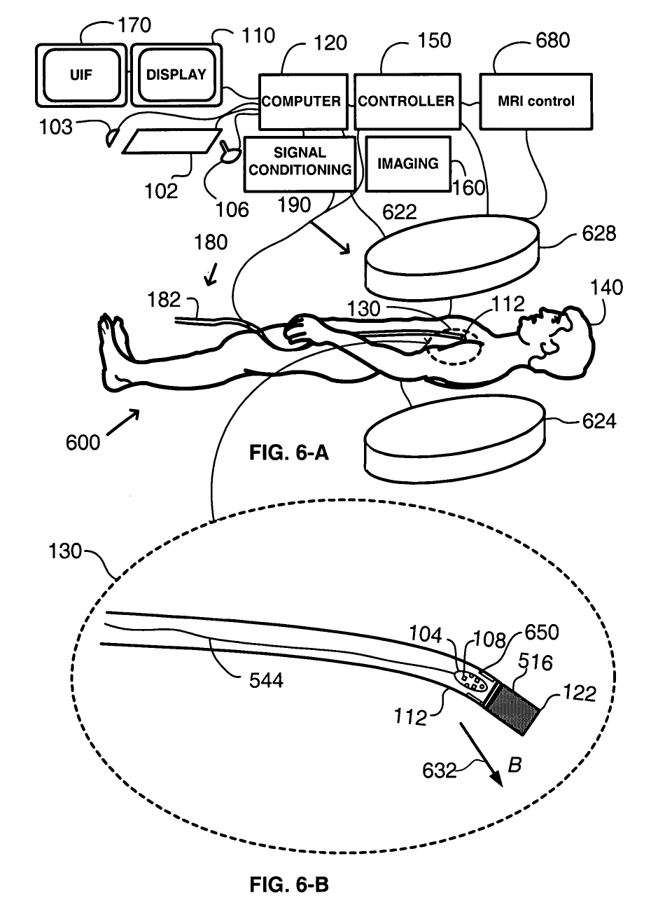

Another system for navigating a medical device is indicated generally in FIG. 6 by reference number 600. A sensing device 104 is provided at or near the tip 122 of a medical device 112. The sensing device includes for example three accelerometers 108 and three gyroscopes 108, each accelerometer being configured to provide an acceleration signal for one of three directional axes. The system 600 is a magnetic navigation system designed for operation within a magnetic resonance imaging (MRI) system and the catheter tip 122 includes one or more magnetic coils 650.

A physician uses an interface 170 and computer 120 to navigate the device tip 122 in a magnetic field 632 produced by one or more magnetic field sources 624, 628. Field source could be a permanent magnet, an electromagnet, a cooled superconducting electromagnet. As described in the context of FIG. 1, the user interface 170 may include a keyboard 102, mouse 104, joystick 106, and/or other device to input instructions to the computer 120. The interface 170 also may include one or more displays 110 whereby the physician may view images provided by the MRI and monitor navigation of the device 112. An MRI apparatus provides a main static, single-direction magnetic field and varying gradient fields in an operating region 130 of the subject. One or more additional magnet(s) (not shown) may be positioned relative to the subject to supplement the MRI field with a navigating field, thereby eliminating or reducing the navigation limitation associated with a main fixed field direction. Alternatively or in addition, the device 112 might comprise “boost magnets” or supplementary electromagnets 650 at or near the device distal end; such additional magnets allow generation of a dipole moment in any direction with respect to the main axis direction at the device distal end, thus facilitating navigation. Further, MRI navigation system 600 might include means to move the subject during the navigation. Additionally, specific design consideration allow gradient imaging coils and/or additional gradient coils to be turned on at high power for extended periods of time and under control of the system computer and controller, so as to facilitate magnetic navigation.

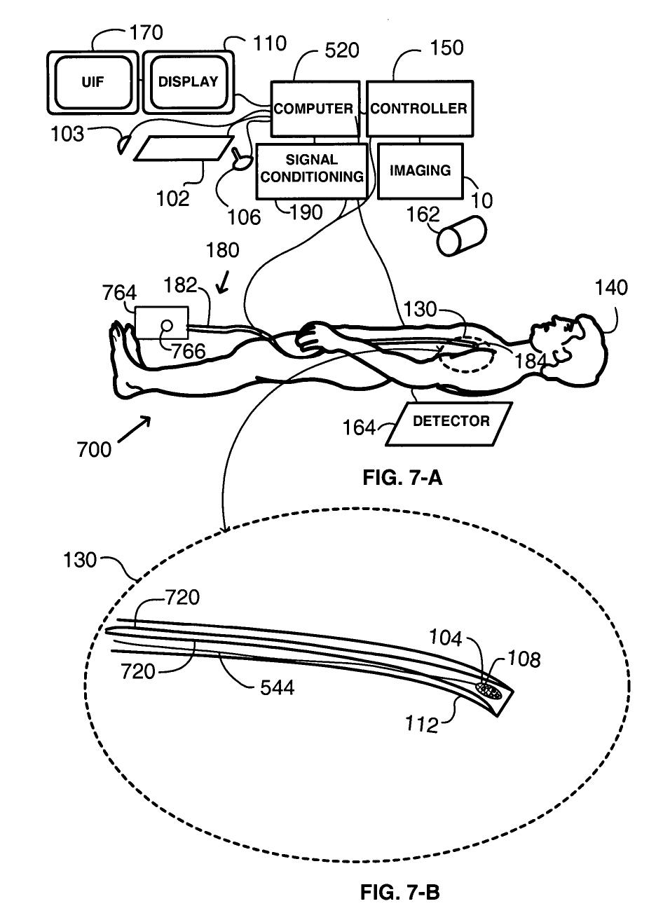

Another embodiment of a navigation system is indicated generally in FIG. 7 by reference number 700. An inertial sensing device 104 is positioned at or near the tip 122 of a catheter 112. The sensing device for example includes three accelerometers 108 and three gyroscopes 108, each accelerometer configured to provide an acceleration signal for one of three directional axes. Signal leads 544 extending through the medical device 112 carry acceleration signals from the inertial sensing component 104 to a computer 120. The computer 120 includes a screen 110 in which may be displayed images of an operating region 130 provided by an imaging apparatus 160. Signals from the inertial sensing device 104 are conditioned by signal processing or conditioning components at 190 and delivered to the computer 120. The signal processing or conditioning components 190 may include a computer with a preconditioning or impedance matching circuit.

Navigation of the catheter 112 is controlled by a physician who uses a manual control wire device 764 to mechanically manipulate the catheter tip 122. Wires 720 or other mechanical elements may be used to control the direction of the catheter tip 122. The wires 720 are attached to a knob 766 and/or levers (not shown) operated by the physician. Action by the physician thus is part of a closed control loop for navigating the catheter 112. Other or additional elements for controlling the catheter 112 may include a gear system run by a flexible shaft, to bend the catheter tip.

In another implementation, the wires 720 may be operated by the computer 120 acting in response to imaging and physician input. In one feedback method in accordance with the invention, the wires 720 can be operated based on the signals from the inertial sensing device 104 through the computer 120 to follow a planned path, or to give a desired location and curve to the catheter 112 if a mechanical catheter model linking known inputs to output responses is available. If desired, real time-physician input can be included in the control loop.

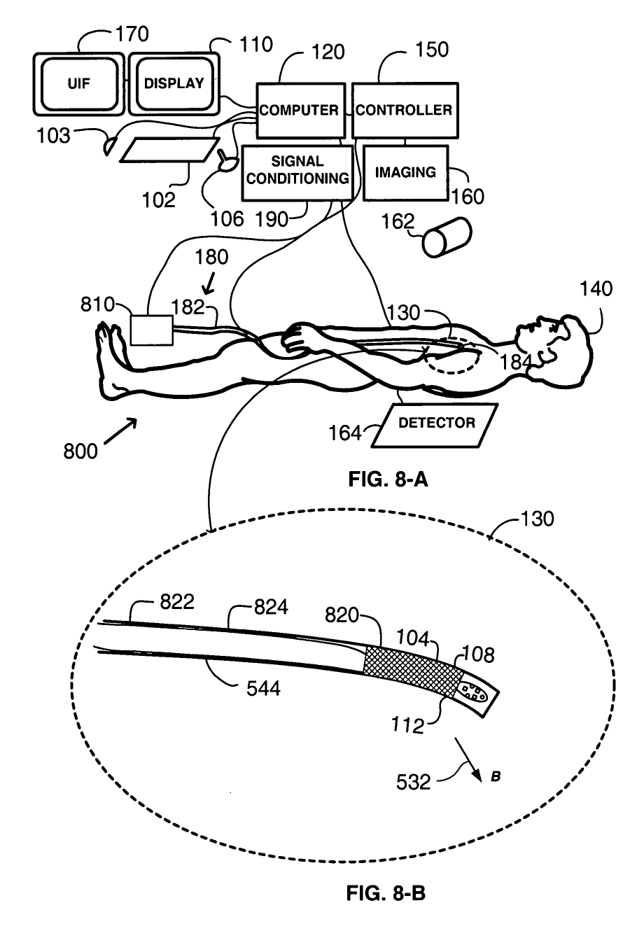

Another embodiment of a navigation system is indicated generally in FIG. 8 by reference number 800. The system 800 may be used to navigate an electrostrictively shaped catheter 112. An inertial sensing device 104 is provided at the medical device tip 122. Signals from the sensing device 104 are carried by leads 544 from the medical device 112 to signal processing or conditioning components at 190. The signals may be processed, for example, as previously described with reference to FIG. 1 and may be further processed by a computer 120.

After being processed in the foregoing manner, the inertial signals may be used to operate a voltage control 810 to control a plurality of electrostrictive elements 820 adjacent a medical device wall 824 to bend and/or guide the tip 122 to move the tip to a desired location and apply a desired force, for example, on a heart wall. A user interface 170 also may be used to receive real-time physician input if desired, e.g., as previously described with reference to FIG. 5.

Advantageously, electrical wires 822 finer than wires typically used for mechanical manipulation can be positioned in the device wall 824 to operate the electrostrictive elements 820, allowing the medical device 112 to be more flexible than one bent by mechanical wires. It is known that electrostriction uses minimal power amounts, and hence small currents, except possibly during a change in configuration.

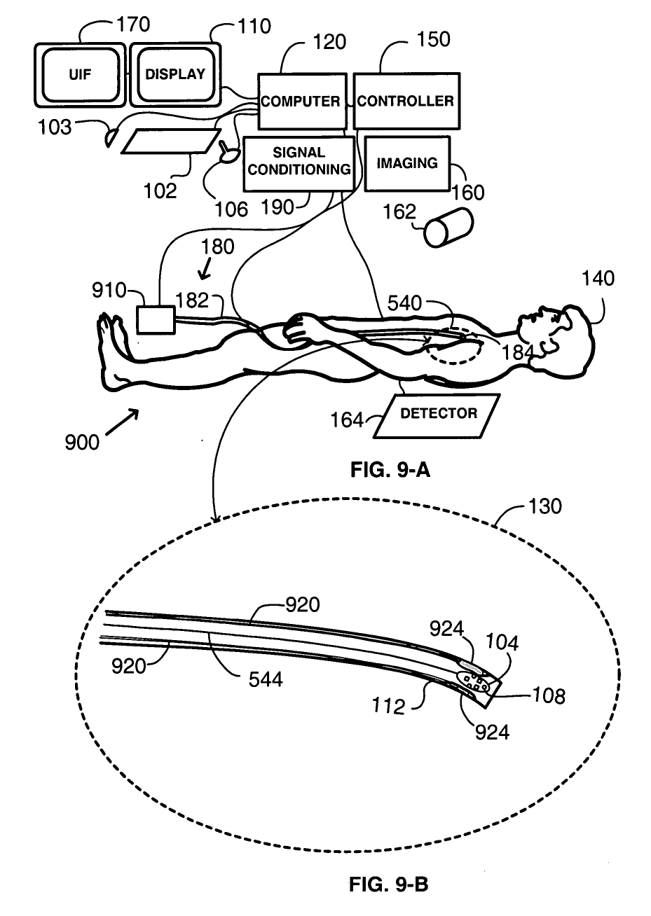

Another embodiment of a system for navigating a medical device is indicated generally in FIG. 9-A by reference number 900. The system 900 can be used to activate a naturally straight catheter 112 by hydraulic means. A plurality of fluid channels 920 extend from a tip 122 of the catheter through the proximal end (not shown) of the catheter. The channels 920 are connected with a fluid control panel 910 preferably located near the subject. One or more of the fluid channels 920 is attached to one or more catheter bend locations, e.g., one or more lengthwise expandable segments 924 of the catheter 112.

An inertial sensing device 104 is positioned at the catheter tip 122. Inertial signals from the sensing device 104 are carried by leads 544 from the catheter 112 to a conditioning block 190. The signals may be processed, for example, as previously described with reference to FIG. 1 and may be further processed by a computer 120. The processed inertial signals may be input to the fluid control panel 910 via a fluid control interface to control fluid in the fluid channels 920 and expansion channels 924. A user interface 170 with the computer 120 also may be used to receive real-time physician input if desired, e.g., as previously described with reference to FIG. 5. The catheter 112 can be bent near the tip 122 by appropriate pressure of fluid acting on one or more of the expansion channels 924 to give a bend in a desired direction. Additionally or alternatively, the system 900 can be used to straighten a naturally pre-bent catheter.

Very little power is needed for inertial sensing at the tip of a medical device; accordingly, fine wires typically are sufficient to provide power to the sensor(s) and to carry the signals back to the conditioning block.

The advantages of the above described embodiment and improvements should be readily apparent to one skilled in the art, as to enabling the navigation of interventional devices within a subject using MEMS inertial devices. Additional design considerations may be incorporated without departing from the spirit and scope of the invention. Accordingly, it is not intended that the invention be limited by the particular embodiment or form described above, but by the appended claims.

What is claimed is:

1. A method of operating a remote navigation system that controls the position of a medical device in an operating region in a subject, the method comprising:
operating the remote navigation system to change the position of the medical device,
processing signals from at least one inertial sensor associated with the medical device to determine the change in position from the initial position;
comparing the determined change in position with the desired position; and
repeating the steps until the current position is within a predetermined value of the desired position.

2. A method of determining the localization information of a medical device being navigated through an operating region in a subject, the method comprising:
processing signals from at least one inertial sensor to estimate the movement of the device;
determining the current localization of the device using the known initial localization of the device and the estimated movement of the device.

3. The method of claim 2, wherein the localization information comprises at least position information.

4. The method of claim 2, wherein the localization information comprises both position and orientation information.

5. The method of claim 2, wherein a set of inertial sensors provides acceleration information along three axes and direction information for each of the three axes with respect to a set of three axes of known directions.

6. The method of claim 5, wherein velocity information is determined along three axes of known orientation from the acceleration information, and wherein position information is determined from initial position and orientation information and knowledge of velocity information with respect to three axes of known time-varying orientation.

7. The method of claim 2, further comprising displaying a representation of the device on an image of the operating region in the neighborhood of the determined localization.

8. The method of claim 1, wherein the actuating medical device controls comprise steering a medical device distal end comprising a magnet by externally generating and applying a magnetic field of specific magnitude and orientation at the device distal end.

9. The method of claim 1, wherein the actuating medical device controls comprise steering a medical device distal tip by applying pull forces on a number of pull wires running internally within the device.

10. The method of claim 1, wherein the actuating medical device controls comprise steering a medical device distal tip by applying forces at the device distal end through hydraulic pressure generated by injecting a fluid at the device proximal end and conducting the fluid through a device lumen to pressure chambers located at the device distal end.

11. The method of claim 1, wherein the actuating medical device controls comprise steering a medical device distal tip by applying voltages to one or several electrostrictive elements located at the device distal end.

12. The method of claim 11, wherein the actuating medical device controls further comprise steering a medical device by applying voltages to one or several electrostrictive elements located along the device length.

13. A system for remotely navigating a medical device in an operating region within a subject, the system comprising:
an inertial sensing device comprising one or more inertial sensors generating time series data sufficient for determination of localization information for the medical device; and
a controller operable to control orientation of the medical device distal tip based on the localization information.

14. The system of claim 13, wherein the localization information determined from the inertial sensors data comprises at least position information.

15. The system of claim 13, wherein the localization information determined from the inertial sensors data comprises both position and orientation information.

16. The system of claim 13, wherein the controller operates a mechanical device operable to move or orient at least a portion of the medical device.

17. The system of claim 16, wherein the mechanical device comprises a set of pull-wires.

18. The system of claim 13, wherein the controller operates a set of one or more electrostrictive elements positioned at the device distal end.

19. The system of claim 13, wherein the controller operates a mechanical device comprising one or more fluid channels extending into the medical device, the controller operable to control fluid in one or more channels to reshape at least a portion of the medical device.

20. The system of claim 19, wherein the one or more fluid channels are attached to one or more expandable segments of the medical device, the controller operable to control fluid in one or more of the channels to bend the medical device in at least one of the one or more expandable segments.

21. The system of claim 13, further comprising one or more external adjustable magnets, and wherein the controller is operable to control the orientation and magnitude of the externally generated magnetic field at least at the medical device distal end by changing the position and orientation of the external magnets.
